SEQUENCE LISTING (4 sequences)

SEQ ID NO: 1 - DNA, 30 bp, Artificial sequence
  Primer
attgaattccgtccgccgtcgcagccgtat

SEQ ID NO: 2 - DNA, 36 bp, Artificial sequence
  Primer
ttagaattccctcccgcccctcgaataaacaacgct

SEQ ID NO: 3 - DNA, 29 bp, Artificial sequence
  Primer
attcactagtgtacggtgggaggtctata

SEQ ID NO: 4 - DNA, 31 bp, Artificial sequence
  Primer
tctagtttaaacaccctaactgacacacatt

(19) United States
(12) Patent Application Publication
(10) Pub. No.: US 2004/0229362 A1
(43) Pub. Date: Nov. 18, 2004

(54) Method for producing non-pathogenic helper virus-free preparations of herpes virus amplicon vectors, the helper virus & the cells used in this method, the corresponding genetic tools, as well as the applications of these non-pathogenic amplicon vectors

(76) Inventors: Alberto Epstein, Lyon (FR); Cecile Zaupa, Bassoues (FR); Carine Logvinoff, Vaulx en Velin (FR)

Correspondence Address: JAMES C. LYDON, 100 DAINGERFIELD ROAD, SUITE 100, ALEXANDRIA, VA, 22314, US

(21) Appl. No.: 10/438,019

(22) Filed: May 15, 2003

(51) Int. Cl. C12N 7/00; C12N 15/86

(52) U.S. Cl. 435/457; 435/235.1

(57) ABSTRACT

A defective Cre-loxP based helper virus (HSV-1 LaLΔJ), which genome is of reduced size and is free of the genes encoding ICP4 and ICP34.5 proteins from the helper genome, in addition to the native “a” signals. HSV-1 LaLΔJ carries a single floxed “a” signal in gC locus. To produce HSV-1 LaLΔJ and to prepare the amplicon vectors, two novel cell lines expressing the essential ICP4 protein, either alone or in combination to Cre recombinase, are also disclosed. These cell lines complement ICP4 while minimizing the probability of generating replication-competent particles. The novel helper system enables production of large amounts of high-titer amplicon vectors. Residual helper particles generated do not exceed 0.5% of the viral population and can grow only in cells expressing ICP4. Amplicon vectors produced with this method showed no cytotoxicity for infected cells.

### FIELD OF THE INVENTION

The field of the invention is the one of the amplicon vectors, notably useful for the gene transfer into a wide variety of cells and of the preparation of these amplicon vectors.

The present invention related to improved methods for making non-cytotoxic helper virus-free preparations of herpes virus amplicon vectors (or particles); to these vectors (or particles) per se; to the means (recombinant helper virus & cells) involved, and to the methods of using these non-cytotoxic helper virus free amplicon vectors (particles) to treat patients, as well as to methods of using these amplicon vectors as tools in therapy (genic therapy—vaccinations), immunology, biology, biotechnology and genetic engineering (cf. abstract).

### BACKGROUND

(N.B: All hereinafter cited prior references that are not patents or patent applications, but articles, are identified at the end of the instant description)

The amplicons vectors concerned by the instant invention derive from herpes viridae species. More particularly, the encompassed sub-species is the one of Herpes simplex virus type 1 (HSV), which is a DNA virus capable of rapidly and efficiently infecting a wide variety of cell types (Leib and Olivo, 1993). Amplicon vectors (plasmid-based viral vectors), are very interesting and promising helper-dependent HSV-1-derived vectors. It remains however difficult and expensive to obtain large amounts of high-titer and non-pathogenic vector stocks, in spite of several important advances in this field.

We known some defective amplicon vectors derived e.g. from herpes simplex virus type 1 (HSV-1). The major interest of such amplicon vectors stems from the fact that they carry no transacting virus genes and consequently do not induce synthesis of virus proteins. Therefore these vectors are non-toxic for the infected cells and non-pathogenic for the inoculated organisms. Another advantage that arises from the lack of virus genes is that most of the virus genome—about 150 kbp—and of the capsid volume can be used to incorporate large foreign DNA (Wade-Martins et al., 2001).

Amplicons are helper-dependent vectors that originate from an amplicon plasmid (Spaete and Frenkel, 1982). These are standard plasmids carrying, in addition to the transgenic sequences, one origin of replication (usually ori-S) and one cleavage-packaging signal (“a”) from the HSV-1 genome. In cells expressing the full set of structural, replication and DNA packaging functions from HSV-1, the amplicon plasmid is amplified by a rolling-circle mechanism into long head-to-tail concatemers that are then cleaved and packaged, up to one genome size, into HSV-1 virions (Kwong and Frenkel, 1985; Bataille and Epstein, 1997). Amplicon vectors are thus a concatemeric plasmid DNA packaged into HSV-1 particles.

Following the initial demonstration that they could be used to deliver foreign DNA (Kwong and Frenkel, 1985), amplicons have been widely and successfully employed for transfer and expression of a variety of genes of neurobiological (Geller and Breakefield, 1988; Ho et al., 1993), immunologic (Willis et al., 2001; Delman et al., 2002; Hocknell et al., 2002) or therapeutic (Federoff et al., 1992; Carew et al., 2001) interest into cultured cells and living organisms.

Until recently, amplicon vector stocks were prepared in cells transfected with the amplicon plasmid and superinfected with helper HSV-1. As the helper virus was generally a replication-defective mutant of HSV-1, the amplicon stocks were produced on transcomplementing cell lines (Geller et al., 1990; Lim et al., 1996). However, the use of HSV-1 as helper resulted in the production of helper-contaminated vector stocks and the contaminating particles, though defective, induced significant cytotoxicity and inflammatory responses (Johnson et al., 1992), thus precluding their use in gene therapy or vaccination protocols.

To overcome these obstacles, novel helper systems that can produce essentially helper-free vector stocks have been recently developed.

The pioneer of these systems was based on the cotransfection of amplicon plasmids with HSV-1 genome fragmented into a set of cosmids (Fraefel et al., 1996; Sun et al., 1999) or more recently with bacterial artificial chromosomes (Saeki et al., 1998; Stavropoulos and Strathdee, 1998; Saeki et al., 2001), that supply most or all of the helper functions, but are deleted of their “a” signals, thus preventing their packaging into HSV-1 virions. Although the more recent versions of these systems do allow production of helper-free amplicons, the amount of the amplicon vector stocks produced in this way are rather low, since they are based on DNA transfection procedures and the vector stocks cannot be further amplified. Therefore, these methods appear expensive and hardly suitable for large-scale production of amplicon vectors.

WO-A-02/056828 (US-A-2003/0027322) discloses 2 methods of generating a herpes virus amplicon particle.

The first method comprises providing a cell that has been stably transfected with a nucleic acid sequence that encodes an accessory protein; and transfecting the cell with (a1) one or more packaging vectors that, individually or collectively, encode one or more HSV structural proteins but do not encode a functional herpes virus cleavage/packaging site and (b) an amplicon plasmid comprising a sequence that encodes a functional herpes virus cleavage/packaging site and a herpes virus origin of DNA replication.

The second method comprises transfecting a cell with (a) one or more packaging vectors that, individually or collectively, encode one or more HSV structural proteins but do not encode a functional herpes virus cleavage/packaging site; (b) an amplicon plasmid comprising a sequence that encodes a functional herpesvirus cleavage/packaging site, a herpesvirus origin of DNA replication, and a sequence that encodes an immunomodulatory protein, a tumor-specific antigen, or an antigen of an infectious agent; and (c) a nucleic acid sequence that encodes an accessory protein.

The herpes virus is an alpha herpes virus (Varicella-Zoster virus, pseudorabies virus, herpes simplex virus), a beta herpes virus or a gamma herpes virus.

The accessory protein, which inhibits gene expression in the cell is a virion host shutoff protein (e.g. from HSV-1, HSV-2, bovine herpes virus 1, bovine herpes virus 1.1, gallid herpes virus 1, gallid herpes virus 2, suid herpes virus 1, baboon herpes virus 2 virion, pseudorabies, cercopithecine herpes virus 7, meleagrid herpes virus 1, equine herpes virus 1, or equine herpes virus 4).

The cell is further transfected with a sequence encoding a VP16 protein, having e.g. the same origin as the virion host shutoff protein.

The packaging vector can be a cosmid, a yeast artificial chromosome, a bacterial artificial chromosome, a human artificial chromosome, or an F element plasmid.

So, this prior reference refers to helper virus-free amplicon packaging methods belonging to the same approach as the hereabove mentioned one (Fraefel et al., 1996; Sun et al., 1999; Saeki et al., 1998; Stavropoulos and Strathdee, 1998; Saeki et al., 2001).

Recently, the inventors have developed an alternative helper system for amplicon production, which is based on the deletion, by site-specific recombination, of the packaging signals of the helper virus in the cells where the vector stock is being produced. This system uses, as helper, a recombinant HSV-1 (named HSV-1 LaL) that carries a unique and ectopic cleavage-packaging “a” signal flanked by two loxP sites in parallel orientation (Logvinoff and Epstein, 2000a). In cells expressing Cre recombinase protein (TE-CRE 30 cells) (Logvinoff and Epstein, 2000b), HSV-1 LaL retains the ability to replicate its DNA and to express early and late viral functions, but remains largely uncleaved and unpackageable due to efficient Cre-induced deletion of the “floxed” “a” signals. As this system is based on infection, instead of on cotransfection procedures, it enables serial passages of the vector stocks, allowing to prepare large amounts of high-titer amplicon stocks. Furthermore, the vector stocks prepared by this method contained only very low levels (lower than 1%) of contaminating helper particles (Logvinoff and Epstein, 2001), indicating that the Cre-loxP site-specific recombination system worked very efficiently in the context of HSV-1 infected cells. However, the few contaminant particles still present in the vector stocks, that result from genomic units that have escaped site-specific deletion of the packaging signals (Logvinoff and Epstein, 2000a), are replication-competent and can thus disseminate in cells not expressing Cre recombinase, preventing their use in humans.

This new Cre-loxP-based approach has been also adopted by the Chinese patent application CN-A-1263159, which concerns a new type simple herpes virus HSV-1 amplicon carrier system in the DNA of which is inserted, between loxP sequences of Cre protein recombinase specific excision and isodirectional arrangement, the HSV-1 packaging signal loxP-pac-loxP whose two sides possess isodirectionally-arranged loxP sequence. the original packaging signal pac being removed, so as to obtain “the recombinant helper virus containing removable packaging signal” rHSV-1/loxP-pac-loxP. Said helper virus only has infection and replication ability in the cell expressing Cre recombinase, does not package out progeny virus. By using rHSV-1/loxP-pac-loxP as helper virus the amplicon virus is produced in the cell expressing Cre recombinase to obtain the goal of reducing helper virus and increasing amplicon virus titer at the same time.

In this state of the art, one of the essential objectives of the invention is to provide easily, cheaply and industrially high quantities of amplicons free (or almost free) of pathogenic and cytotoxic helper virus, in order to avoid the risks associated with such potential dissemination of helper particles, that could occur during the uses, notably the therapeutics uses (gene therapy and vaccines) of the amplicon vectors.

Another essential objective of the invention is to improve the Cre-loxP-based approach in the production of amplicons, by providing an improved and optimized amplicon vectors production method, as well as a second-generation, defective and non-pathogenic, preferably Cre-loxP based helper system, which is significantly safer than the (Logvinoff and Epstein, 2001) system based on the use of HSV-1 LaL.

Another essential objective of the invention is to propose several significant improvements to the previously described, Cre-loxP-based approach, to generate high amounts of amplicon vectors with only very low levels of contamination with helper particles (e.g. lower than 0.5%).

Another essential objective of the invention is to provide novel defective helper virus, that allow high production of vectors without generating replication-competent particles.

Another essential objective of the invention is to provide novel cell lines, that allow high production of vectors without generating replication-competent particles.

Another essential objective of the invention is to provide recombinant genetic tools which would enable to realize the means composing the system implemented in the invention, and notably in the non-pathogenic amplicon vectors production.

Another essential objective of the invention is to provide performing method for the construction of the virus helper and the cell lines involved in the present invention.

Another essential objective of the invention is to provide new uses (particularly in therapy and prevention—gene therapy—, in vaccines, in genetic engineering and in biotechnology) for the amplicon vectors prepared by the method and with the means according to the invention.

### SUMMARY

These objectives, among others, have been reached by the inventors who had the merit to find out:

that the defective helper virus, on the one hand, should carry deletions—preferably in two virus loci—in order to reduce virulence, and on the other hand, should be subjected to a significant genomic size reduction, in order to prevent encapsidation and so development of the helper virus;

that this defective helper virus should be combined with at least two cell lines conceived in order to efficiently transcomplement the deleted protein minus helper, while minimizing the probability of homologous recombination at the locus where the deletions have been made.

Thus, according a first of its aspects, the invention concerns a method for producing non-pathogenic defective amplicon vectors derived from herpes viridae species by means of an helper system comprising at least one kind of cells and at least one kind of helper virus which is finally at least partially deleted by means of a site-specific recombination system involving the packaging signals “a” of the helper virus in the cells where the amplicon vectors are produced,

said method including notably the following essential steps:

-a- transfection of cells C1 from a first cell line by the amplicon vectors;

-b- (super)infection of said cells C1 with the helper virus;

-c- culture of transfected and (super)infected cells C1;

-d- harvest of the so produced amplicon vectors and helper virus;

-e- infection of cells C2 from a second cell line different from C1, by at least one part of the harvested amplicon vectors and helper virus;

-f- culture of infected cells C2;

-g- harvest of the so produced particles of the amplicon vectors free or substantially free of helper virus;

wherein:

(i) the helper virus's recombinant genome has preferably a size S (kbp) defined as follows with respect to the reference size Sr (kbp) of the virus's helper genome free from any deletion of coding sequence(s) encoding for at least one protein essential for viral production of the helper virus:

| | |
|---|---|
| | S ≦ 0.99 · Sr |
| preferably | S ≦ 0.95 · Sr |
| more preferably | S ≦ 0.90 · Sr |

(ii) the helper virus's recombinant genome includes a packaging specific site recognizable and deletable by cells C2;

(iii) the infection -e- of cells C2 by the helper virus results in deletion of the packaging signal(s) “a”, said deletion so involving an additional size reduction;

(iv) the helper virus's recombinant genome is totally or partially defective in coding sequence(s) encoding for at least one essential protein (Pe) and eventually at least one non-essential protein (Pne) for viral production of the helper virus;

(v) the cells C1 and C2 are able to transcomplement at least one of the essential protein(s) Pe and eventually at least one of the non-essential protein(s) (Pne) and are so able to make up for the genomic deficiency of the helper virus;

(vi) and the cells C2 are able to recognize and to delete the packaging specific site “a” of the helper virus.

### BRIEF DESCRIPTION

The new and inventive method of the invention is notably based on the implementation of a novel system is composed of at least three elements:

(i) a defective helper virus, in particular the one named HSV-1-LaLΔJ, which contains at least one—preferably at least two—independent safety barriers, as it lacks the genes encoding one essential protein Pe (e.g. ICP4) and another protein Pne which is non-essential, e.g. the neurovirulence factor ICP34.5

(ii) a novel complementary cell line expressing one of the lacking protein(s) (e.g. ICP4) (said cell line comprising e.g. BHK-CINA6 cells) and

(iii) a cell line expressing both one of the lacking protein(s) (e.g. ICP4) and (enzymatic) means capable of deleting the packaging signals, for example Cre recombinase (said cell line comprising e.g. TE CRE GRINA129 cells).

These two cell lines are conceived in order to efficiently transcomplement the helper lacking essential protein(s) (e.g. ICP4), while minimizing the probability of homologous recombination at the deletion locus.

The invention includes a safer and efficient helper system that allows easy production of high amounts of non-pathogenic amplicon vectors. Amplicon vectors produced by this way are non cytotoxic for the infected cells. The residual helper particles still present in the vector stocks are defective and cannot spread in standard cell lines or in vivo organisms.

According to the invention the qualifier “essential” in the expressions “essential or non-essential proteins or locus”, means that the given protein or locus is essential (or not) for the life of all kinds of cells.

According to a preferred feature of the protocol to vector production of the invention, the helper virus's recombinant genome is subjected

to a first size reduction corresponding to the deletion of the coding sequence(s) encoding for at least one protein essential (Pe) and eventually at least one non-essential protein (Pne) for viral production of the helper virus, said first size reduction occurring before cells C1 & C2 (super)infections,

and to second size reduction corresponding to the deletion of the packaging specific site “a” of the helper virus, in the cells C2;

so that the helper virus encapsidation be prevented.

In the best way of implementation of the invention's method, the site-specific recombination system involving the packaging signals “a” of the helper virus, comprises at least one enzyme specific of at least one sequence delimited by 2 identical sites, said system being preferably selected in the group including enzyme Cre/sites loxP-“a”-loxP and enzyme Flp/sites frp-“a”-frp.

The construction and properties of said preferred defective helper system for packaging amplicon vectors, which is considerably safer and more efficient than the previously published HSV-1 LaL/TE CRE 30 system (Logvinoff and Epstein, 2001). The novel system which, like the previous one, is based on site-specific deletion of the unique, loxP sites surrounded “a” packaging signal, in cells expressing the Cre recombinase, is composed of three elements that have been constructed according to the instant invention. According to a remarkable feature, the helper virus's recombinant genome contains at least one (preferably a single) floxed “a” packaging signal located in non-essential loci, preferably in gC locus.

Preferably, the missing proteins of the helper are at least two of them and at least one is essential.

Concerning the neutralization of their encoding sequences, the deletion can be total or partial. Then, at least part of the coding sequence(s) encoding for essential protein (Pe₁) and one non-essential protein (Pne₁) are lacking in the helper virus's recombinant genome, Pe₁ and Pne₁ being preferably selected in the ICP proteins group, and more preferably Pe₁ being ICP4 and Pne₁ being ICP34.5.

According to an interesting aspect of the invention's method, the final residual virus helper particles concentration is inferior or equal to 0.5%, preferably to 0.3%, and more preferably to 0.2% of the produced viral population.

Preferably, Sr is comprised between 10 to 500 kbp, preferably between 50 to 300 kbp, and more preferably between 100 to 200 kbp.

Without any limitation, the amplicon plasmid contains at least one gene of neurobiological, immunologic or therapeutic interest.

The compositions obtained by the production method of the present invention (including herpes virus particles and cells that contain them) can be used to treat patients who have been, or who may become, infected with a wide variety of agents (including viruses such as a human immunodeficiency virus, human papilloma virus, herpes simplex virus, influenza virus, pox viruses, bacteria, such as E. coli or a Staphylococcus, or a parasite) and with a wide variety of cancers. A patient can be treated after they have been diagnosed as having a cancer or an infectious disease or, since the agents of the present invention can be formulated as vaccines, patients can be treated before they have developed cancer or contracted an infectious disease. Thus, “treatment” encompasses prophylactic treatment.

As noted, the herpes viridae amplicon particles described herein (and the cells that contain them) can express a heterologous protein (i.e., a full-length protein or a portion thereof (e.g., a functional domain or antigenic peptide) that is not naturally encoded by a herpesvirus). The heterologous protein can be any protein that conveys a therapeutic benefit on the cells in which it, by way of infection with an herpes viridae amplicon particle, is expressed or a patient who is treated with those cells.

The therapeutic agents can be immunomodulatory (e.g., immunostimulatory) proteins (as described in U.S. Pat. No. 6,051,428). For example, the heterologous protein can be an interleukin (e.g., IL-1, IL-2, IL-4, IL-10, or IL-15), an interferon (e.g., IFN.gamma.), a granulocyte macrophage colony stimulating factor (GM-CSF), a tumor necrosis factor (e.g., TNF.alpha.), a chemokine (e.g., RANTES, MCP-1, MCP-2, MCP-3, DC-CK1, MIP-1.alpha., MIP-3.alpha., MIP-.beta., MIP-3.beta., an alpha. or C—X—C chemokine (e.g., IL-8, SDF-1.beta., 8DF-1.alpha., GRO, PF-4 and MIP-2). Other chemokines that can be usefully expressed are in the C family of chemokines (e.g., lymphotactin and CX3C family chemokines).

Intercellular adhesion molecules are transmembrane proteins within the immunoglobulin superfamily that act as mediators of adhesion of leukocytes to vascular endothelium and to one another. The vectors described herein can be made to express ICAM-1 (also known as CD54), and/or another cell adhesion molecule that binds to T or B cells (e.g., ICAM-2 and ICAM-3).

Costimulatory factors that can be expressed by the vectors described herein are cell surface molecules, other than an antigen receptor and its ligand, that are required for an efficient lymphocytic response to an antigen (e.g., B7 (also known as CD80) and CD40L). When used for gene therapy, the transgene encodes a therapeutic transgene product, which can be either a protein or an RNA molecule. Therapeutic RNA molecules include, without limitation, antisense RNA, and an RNA ribozyme. The RNA ribozyme can be either cis or trans acting, either modifying the RNA transcript of the transgene to afford a functional RNA molecule or modifying another nucleic acid molecule. Exemplary RNA molecules include, without limitation, antisense RNA, ribozymes to nucleic acids for huntingtin, alpha synuclein, scatter factor, amyloid precursor protein, p53, VEGF, etc.

Therapeutic proteins include, without limitation, receptors, signaling molecules, transcription factors, growth factors, apoptosis inhibitors, apoptosis promoters, DNA replication factors, enzymes, structural proteins, neural proteins, and histone or non-histone proteins. Exemplary protein receptors include, without limitation, all steroid/thyroid family members, nerve growth factor (NGF), brain derived neurotrophic factor (BDNF), neutotrophins 3 and 4/5, glial derived neurotrophic factor (GDNF), cilary neurotrophic factor (CNTF), persephin, artemin, neurturin, bone morphogenetic factors (B M1's), c-ret, gp 130, dopamine receptors (D 1D5), muscarinic and nicotinic cholinergic receptors, epidermal growth factor (EGF), insulin and insulin-like growth factors, leptin, resistin, and orexin. Exemplary protein signaling molecules include, without limitation, all of the above-listed receptors plus MAPKs, ras, rac, ERKs, NFK.beta., GSK3.beta., AKT, and P13K. Exemplary protein transcription factors include, without limitation, .about.300, CBP, HIF-1alpha, NPAS1 and 2, HIF-1.beta., p53, p73, nurr 1, nurr 77, MASHs, REST, and NCORs. Exemplary neural proteins include, without limitation, neurofilaments, GAP-43, SCG-10, etc. Exemplary enzymes include, without limitation, TH, DBH, aromatic amino acid decarboxylase, parkin, unbiquitin E3 ligases, ubiquitin conjugating enzymes, cholineacetyltransferase, neuropeptide processing enzymes, dopamine, VMAT and other catecholamine transporters. Exemplary histones include, without limitation, H1-5. Exemplary non-histones include, without limitation, ND10 proteins, PML, and HMG proteins. Exemplary pro- and anti-apoptotic proteins include, without limitation, bax, bid, bak, bcl-xs, bcl-xl, bcl-2, caspases, SMACs, and IAPs.

The enabled possible therapeutical treatments which are available thanks to the invention include notably vaccinations.

In practice and for example:

the amplicon plasmid is pA-MuCMV-LacZ,

the helper virus is HSV-1 LaLΔJ,

C1 are BHK-CINA6 cells and

C2 are TE CRE GRINA129 cells,

as defined in the instant specification and enclosed figures.

These are a novel helper virus, which has been named HSV-1 LaLΔJ, and two novel complementing cell lines that express ICP4, either alone (BHK CINA6 cells), or in combination with the Cre recombinase (TE CRE GRINA129 cells).

In the methods according to the invention, the cells C1 (e.g. BHK CINA6) are employed to prepare the passage of helper-contaminated vectors, after transfection of the amplicon plasmid and superinfection with the helper virus. If required, these cells C1 can be used also to make further serial passages in order to amplify the amount of vector contaminated stocks.

The cells C2 (e.g. TE-CRE GRINA129) are used only to prepare the final amplicon stocks using aliquots of the stocks produced on C2 cells (e.g. TE-CRE GRINA129).

After the steps -a,b,c- on cells C1 (e.g. BHK CINA 6), the vector stocks shows a vector to helper ratio varying from 2 to more than 10. Although this ratio can vary both with the batch of amplicon plasmid and with the passage number of cells C1 (e.g. BHK CINA 6), it is always favorable to the amplicon particles. This observation contrast dramatically with vector stocks prepared according to the prior art methods, which always yield helper particles that are largely in excess to those of amplicon particles (vector to helper ratio: 1:10 to 1:50).

Using the invention's helper virus (e.g. HSV-1 LaL), the vector to helper ratio after the first steps -a,b,c-, is also favorable to the helper particles (vector to helper ratio: 1:2 to 1:5). This indeed represents a very favorable situation, as large amounts of good-titer amplicon particles can be easily obtained from the very first passage.

The advantageous properties in favor to the novel system could be notably related to the reduced size of the invention's helper virus (e.g. HSV-1 LaL) genome (about 144 kbp). After the steps -e,f- of the invention's protocol, the vector to helper ratio generally exceeds 200, and sometimes reach 500 (Table 1 and FIG. 5), while this ratio is generally lower than 100 in the prior art. The difference between the ratios observed after the steps -a,b,c- and the steps -e,f- is explained by a dramatic fall in HSV-1 LaLΔJ helper titers, whereas amplicon titers are not significantly affected (they fall between 3 to 6 times as compared to the titers after the the steps -a,b,c-).

The low level of contaminating helper particles present in the vector stocks do not represent revertant genomes, as they are unable to grow further in cells C2 (e.g.TE-CRE GRINA 129). Most likely without being linked to the theory, these particles represent genomic units that have escaped to site-specific deletion of the “a” sequence and were thus packaged, but without gaining the ability to generate virus stocks in cell lines not expressing one essential protein (e.g. ICP4) or expressing (enzymatic) means for deleting packaging signals (e.g. Cre recombinase).

The non-cytotoxic character of amplicon vectors produced using the method and so the system of the invention has been established, as it will result from the examples infra. These results indicate that said vector stocks are virtually non toxic for the infected cells, even if they still contain some contaminant helper particles.

Large amounts of high-titer non-pathogenic and non-cytotoxic amplicon vectors, as more than 1×10⁸ total transducing units could be so easily produced using. These vector stocks present a level of contamination with helper particles that does not exceed 0.5% of vector transducing units. Furthermore, this very low level of contaminant particles are defective, cannot propagate in cells not expressing one or several essential proteins (e.g. ICP4). There are thus 2 safety barriers at the level of neurovirulence.

It represents a significant development in the way to generate amplicon vectors able to be used in experimental gene therapy and vaccination protocols.

Regarding the use aspects, the present invention has also as subjects:

a method of treating a patient comprising administering to the patient an HSV amplicon vectors obtained by the method as herein defined;

a method of treating a patient comprising administering to the patient the infected cells as herein defined; and

drugs for gene therapy comprising the HSV amplicon vectors obtained by the method as herein defined.

According to another of its aspects, the invention encompasses the perfecting helper virus per se which are defective helper virus belonging to herpes viridae species, notably useful for producing non-pathogenic defective amplicon vectors derived from herpes viridae species, said virus comprising a recombinant genome:

(i) which size S (kbp) is preferably defined as follows with respect to the reference size Sr (kbp) of the virus's helper genome free from any deletion of coding sequence(s) encoding for at least one protein essential for viral production of the helper virus:

| | |
|---|---|
| | S ≦ 0.99 · Sr |
| preferably | S ≦ 0.95 · Sr |
| more preferably | S ≦ 0.90 · Sr |

(ii) including a packaging specific site recognizable and deletable by appropriated cells named C2;

>(iii) and being totally or partially defective in coding sequence(s) encoding for at least one protein essential (Pe) for the production of the helper virus.

It is preferable that the defective helper virus's genome comprises at least one sequence including the packaging signals “a” flanked by 2 identical sites, these latter being selected in the group including sites loxP and sites frp, said sequence being specifically attacked by an enzyme selected in the group including Cre and Flp.

More particularly, said recombinant genome contains at least one (preferably a single) floxed “a” packaging signal located in a non-essential locus, preferably in gC locus.

As already mentioned in the frame of the description concerning the invention's method, at least part of the coding sequence(s) encoding for one essential protein (Pe₁) and one non-essential protein (Pne₁) are lacking in the helper virus's recombinant genome, Pe₁ and Pne₁ being preferably selected in the ICP proteins group, and more preferably Pe₁ being ICP4 and Pne₁ being ICP34.5.

Preferably, the defective helper virus according to claim 11, wherein Sr is comprised between 10 to 500 kbp, preferably between 50 to 300 kbp, and more preferably between 100 to 200 kbp.

In practice and for instance, the defective helper virus consists of HSV-1 LaLΔJ, as defined in the instant specification and enclosed figures.

According to still another of its aspects, the invention encompasses recombinant genome of the above described defective helper virus, its transcription products and its traduction products.

The invention also includes the cells C1 or C2 per se, these latter being able to transcomplement at least one of the essential protein(s) Pe of the defective helper virus according to the instant invention and are so able to make up for the genomic deficiency of said defective helper virus.

In these cells C1 or C2, there are preferably one essential protein Pe₁ and one non-essential Pne₁, Pe₁ and Pne₁ being preferably selected in the ICP proteins group, and more preferably Pe₁ being ICP4 and Pne, being ICP34.5.

It must be noted that cells C2 are able to recognize and to delete the packaging specific site “a” of the helper virus.

In practice and for instance, cells C1 consist of BHK-CINA6 cells, as defined in the instant specification and enclosed figures.

In practice and for instance, cells C2 consist of TE CRE GRINA129 cells, as defined in the instant specification and enclosed figures.

According to still another of its aspects, the invention encompasses recombinant genome of the above described cells, their transcription products and its traduction products.

The transfected cells C1 and/or (super)infected cells C1 and the infected cells C2 obtained by the method according to the instant invention, constitute other subjects of the invention, as well as the helper system for producing non-pathogenic defective amplicon vectors derived from herpes viridae species, said system comprising at least one defective helper virus as above defined, cells C1 and cells C2 as above defined.

Regarding the construction of the helper virus, the invention is also directed to the production method of a defective helper virus belonging to herpes viridae species, notably useful for producing non-pathogenic defective amplicon vectors derived from herpes viridae species, consisting essentially in:

I—constructing a recombinant genome:

free from any native packaging specific site “a”

including a packaging specific site recognizable and deletable by appropriated cells named C2;

II—and reducing the size of the genome so as to obtain a size S which contributes at least partially to prevent the helper virus encapsidation.

Preferably:

the construction step -I- consists essentially in:

deleting the native packaging specific site “a” of the helper virus,

inserting into the helper virus genome a single floxed “a” packaging signal located in non-essential loci, preferably in gC locus, said packaging signal “a” being flanked by 2 identical sites, these latter being selected in the group including sites loxP and sites frp, said sequence being specifically attackable by an enzyme selected in the group including Cre and Flp,

and the size reduction step -II- consists essentially in deleting in the recombinant genome, at least part of the coding sequence(s) encoding one essential protein Pe₁ and one non-essential protein Pe₁, Pe₁ and Pne₁ being preferably selected in the ICP proteins group, and more preferably Pe₁ being ICP4 and Pne₁ being ICP34.5, so that the size S (kbp) of the recombinant genome be defined as follows with respect to the reference size Sr (kbp) of the virus's helper genome free from any deletion of coding sequence(s) encoding for at least one protein essential for viral production of the helper virus:

| | |
|---|---|
| | S ≦ 0.99 · Sr |
| preferably | S ≦ 0.95 · Sr |
| more preferably | S ≦ 0.90 · Sr. |

According to a non-limitative example which is described infra in details in the examples, HSV-1 LaLΔJ is e.g. constructed by homologous recombination of a set of cosmids (Cunningham and Davison, 1993) that are modified in order to contain one floxed “a” sequence into the gC locus of cosmid cos56 (giving cos56LaL). In addition a large AseI-XmnI sequence, spanning the native “a” locus and surrounding sequences at either side, in cosmids cos6 and cos48 (giving cos6ΔJ and cos48ΔJ) is deleted. As a consequence, the resulting HSV-1 LaLΔJ virus lacks, in addition to the native “a” signals, the complex locus encoding ICP34.5 protein, ORF 0 and ORF P, as well as most of the sequences encoding the essential ICP4 protein. This illustrative virus is predicted to encode a peptide, containing the first 430 amino acids of ICP40. The virus behaves as an authentic ICP4 minus virus (FIG. 3 and FIG. 4). This virus is also deficient for gC and lacks the 3′ half of minor LAT transcripts as well as the whole set of L/S transcripts. In addition to the floxed “a” signal that was integrated at the XbaI site of gC locus, the virus carries a minigene conferring resistance to zeomycin under the control of the EM7 promoter, that was introduced at the AseI site, just upstream from the IE1 promoter, encoding ICP0. The name of ΔJ, given to this new virus, stems from the fact that its genome lacks a large part of the repeated junction sequences separating the L and S unique components of HSV-1 genomes. The size of the HSV-1 LaLΔJ genome is 144 kbp. In spite of its small size, of the many modifications carried by its genome, and of the fact that the packaged genome has a peculiar configuration, as its ends map to the gC locus (see Logvinoff and Epstein, 2000a), this virus grows rather well on cell lines expressing ICP4, giving titers greater than 10⁸ PFU/ml in BHK-CINA6 cells.

It is then clear that HSV-1 LaLΔJ ICP4 has minus and Cre sensitive phenotype.

Regarding the construction of the cell lines C1 and C2, it is performed by means of classical protocols of transfection known by the skilled man in the art. It must be also emphasized that, in order to minimize any potential homologous recombination between the ICP4 gene integrated in cellular chromosomes and the defective helper virus genome, BHK-CINA6 and TE-CRE GRINA129 cell lines were conceived in such a way that they contain no virus sequences other than the ORF encoding ICP4. Generation of revertant viruses encoding a functional ICP4, is so prevented.

Other features and advantages of the invention will be apparent from the following detailed description & examples.

### LEGENDS OF THE DRAWINGS

FIG. 1.

Construction of HSV-1 LaLΔJ. A) Schematic representation of HSV-1 genome. The expanded map represent the repeated regions encompassing the α4 gene, “a” sequence, the complex locus encoding γ34.5 gene, ORF P and ORF O, α0 gene, and minor LAT ARN.

AseI and XmnI restriction sites used to delete part of these regions are also indicated. B) Principle of HSV-1 LaLΔJ virus construction. Cosmids cos6ΔJ and cos48ΔJ (each carrying a Zeo gene at the place of the deleted AseI-XmnI fragment), were cotransfected with cos56LaL (containing the “a” sequence flanked by two loxP sites), cos14 and cos 28 in cells expressing ICP4. Three days later emerging virus were plaque-purified and amplified. C) Schematic genomic structure of expected HSV-1 LaLΔJ recombinant.

FIG. 2.

A) Schematic representation of HSV-1 cos17+, HSV-1 LaL and HSV-1LaLΔJ genomes. Expanded maps of the regions containing α0, α4, γ34.5 and “a” sequences are shown. The BamHI restriction maps and probes used for Southern blot analysis are also indicated. B) Autoradiographic images of BamH1-digested DNAs of HSV-1 17+, HSV-1 LaL and HSV-1LaLΔJ, hybridized with “a” probe (B1) α0 probe (B2) and a4 probe (B3). Asterics indicate genomics ends of HSV-1 LaL and HSV-1 LaLΔJ. Empty circles correspond to undigested DNA.

FIG. 3.

Analysis of immediate early end late viral polypeptides in infected BHK-21 and BHK CINA6 cells. BHK-21 and BHK CINA6 cells were mock infected or infected at a MOI of 10 pfu/cell with the indicated viruses, and collected at 20 h post-infection. Lysate of these cells were then used to perform Western blots. Proteins were revealed with antibodies specific for ICP4, ICP0, ICP34.5 and US11.

FIG. 4.

BHK-CINA 6 cells efficiently transcomplement HSV-1 LaLΔJ. Confluent BHK-CINA6 cells, BHK-21 cells and M64A cells, seeded in 60-mm-diameter tissue culture dishes, were infected at an MOI of 0.1 with HSV-1 LaLΔJ. Two days later, infections were stopped and virus titers were estimated by plaque assay on E5 and VERO cell monolayers. All results are average from two experiments; bars indicate the standard deviation.

FIG. 5.

TE CRE GRINA129 cells express ICP4 and Cre proteins

Confluent BHK-CINA6 and TE CRE GRINA 129 cells, seeded in 60-mm-diameter tissue culture dish, were infected with 0.1 MOI of HSV-1 D30EBA or HSV-1LaL. Two days later, infections were stopped and virus titers were estimated by plaque assay on E5 cell monolayers.

All results are average from two independent experiments; bars indicate the standard deviation.

FIG. 6.

Protocole to produce amplicon vectors in two steps. The first step corresponds to amplification of both amplicon vectors and helper viruses, in the classical way, i.e, by superinfection with HSV-1 LaLΔJ of BHK-CINA6 cells (ICP4 expressing cells) transfected by amplicon plasmid. The second step consists of infecting TE-CRE GRINA129 cells (ICP4 and Cre recombinase expressing cells) with the previous production (amplicon vectors and helper viruses). The Cre recombinase induces deletion of the cleavage-packaging “a” sequence of HSV-1 LaLΔJ virus. However, the helper genome is expressed and replicated allowing the production of amplicon vectors only. Two days later, viral stocks were collected.

FIG. 7.

Effects of amplicons and HSV-1 LaLΔJ MOI in the production of amplicon vector on TE CRE GRINA 129 cells. Confluent TE CRE GRINA129 cells, seeded in 60-mm-diameter tissue culture dishes, were infected at an MOI of 0.5 (A), 1 (B), 2.5 (C) and 5 (D) of amplicon vector and with different MOI of HSV-1 LaLΔJ helper virus. When necessary, HSV-1 LaLΔJ virus was added in order to obtain the desired MOI. Two days later, particles were collected after cell sonication and titrated. ▪ represent amplicon titers (TU/ml), □ helper titers (PFU/ml) and -▴- ratio amplicon/helper output.

FIG. 8.

Viability and expression of amplicon infected cells. 2×10⁵ G16.9 cells seeded in 24 well plaque were either mock infected (A) or infected with amplicon stock produced in BHK CINA6 (B), or in TE CRE GRINA 129 (C) cell lines. All infection were made at a MOI of 5 amplicon vectors per cell. Two days post-infection, cells were trypsinized, pelleted, and resuspend in PBS supplemented with 1 □g/ml of propidium iodide (PI). Then cells were analysed by flow cytometer in order to determine dead cells (PI-fluorescence-FL3-H) and transduced cells (GFP-fluorescence-FL1-H). Mock infected cells served to set quadrant border. Cells in the upper left (UL) quadrant are GFP negative and PI positive. Cells in the lower left (LL) quadrant are GFP negative and PI negative. Cells in the upper right (UR) quadrant are GFP positive and PI positive. Cells in the lower right (LR) quadrant are GFP positive and PI negative. Quadrant values (%) are shown in the table below each plot.

Table 1. Titers of Amplicons Vectors and Helpers Particules Prepared Using the Novel System

Abbrevation: Avg.: average

^{a} Sub-confluent BHK CINA6 cells in 60-mm-diameter tissue culture dishes were transfected with 1 μg of pA-MuCMV-LacZ amplicon DNA. One day later, cells were superinfected with HSV-1 LaLΔJ virus at an MOI of 0.25. Cells were incubated an additional two days at 34° C. and particles were collected and titrated.

^{b} Confluent TE CRE GRINA129 cells, seeded in 60-mm-diameter tissue culture dish, were infected with the amplicon/helper stocks produced on BHK-CINA6 at a MOI of 1 amplicon vectors per cell Two days later particles were collected and titrated.

^{c} titers of amplicon vectors were determined on Gli36 cell monolayers by counting blue cells after X-gal staining 24 h.

^{d} titers of helper virus were determined on E5 cell monolayers by counting plaques at 72 h postinfection.

^{e} Independent experiments

### DETAILED DESCRIPTION AND EXAMPLES

.1. Materials and Methods

Cell Lines and Viruses

Vero (African green monkey kidney), E5 (Vero-derived cell line expressing ICP4 protein) (DeLuca et al., 1985), TE-CRE 30 (TE-671-derived cells expressing Cre recombinase) (Logvinoff and Epstein, 2000a), Gli36 (a human glioblastoma kindly obtained from Dr. D. Louis, Harvard, Mass., USA) (Kashima et al., 1995) and G16.9 (Gli36-derived cells expressing VP16, unpublished material kindly obtained from by Dr Y. Saeki, Harvard, Mass., USA) cells were propagated in Dulbecco's minimum essential medium (DMEM) (Invitrogen, Paisley, UK) supplemented with 10% fetal bovine serum (FBS) (Invitrogen), penicillin (100 U/ml) and streptomycin (100 μg/ml) (Invitrogen). BHK-21 (baby hamster kidney) and M64A (BHK-21-derived cells expressing ICP4) (Davidson and Stow, 1985) cells were propagated in DMEM supplemented with 10% FBS, 10% tryptose phosphate broth (TPB) (Sigma Aldrich, St Louis, Mo., USA), penicillin (100 U/ml) and streptomycin (100 μg/ml). All cell lines were maintained at 37° C. in humidified incubators containing 5% CO₂.

The virus named HSV-1 cos17+was obtained by cotransfection of BHK-21 cells with overlapping HSV-1 sequences carried by cosmid set C, as previously described (Cunningham and Davison, 1993). The resulting virus was grown and titrated in Vero cells. HSV-1-LaL (Logvinoff and Epstein, 2000b) was grown in BHK-21 cells and titrated in Vero cells. The ICP4 minus HSV-1 D30EBA (Paterson and Everett, 1990) was grown and titrated in M64A cells.

Plasmids and Cosmids

Construction of pGem ICP4

The α4 open reading frame (ORF) encoding ICP4 was amplified by PCR from cos6 using primers ATT GAA TTC CGT CCG CCG TCG CAG CCG TAT and TTA GAA TTC CCT CCC GCC CCT CGA ATA AAC AAC GCT (EcoRI sites are underlined). These primers correspond to nucleotides 147058 to 147078 and 151104 to 151079 respectively of the HSV-1 genome. PCR was carried using the kit GC Rich PCR system (Roche, Indianapolis, Ind., USA) according to the manufacter's protocol. The 4 kbp PCR product was introduced into pGemT (Promega, Madison, Wis., USA), generating the plasmid pGemICP4.

Construction of pCINA

The α4 ORF was subcloned from pGemICP4 into pIRESNeo2 (Clontech, Palo Alto, Calif., USA) using EcoRI sites. The plasmid with the expected orientation, named pCINA, contains the α4 ORF under the control of the human cytomegalovirus (HCMV) major immediate early promoter, followed by the internal ribosomal entry site (IRES) of encephalomyocardis virus (ECMV), the neomycin phosphotransferase ORF, conferring G418 resistance, and the polyadenylation signal of bovine growth hormone (BGH).

Construction of pGRINA

The plasmid pGRINA was generated as follows. The 4 kbp EcoRI-MseI fragment of pGemICP4, containing α4 ORF, was inserted into the multiple cloning site of pIRESNeo2 (Clontech) between the EcoRI and the NotI sites, after blunt-ending of MseI and NotI sites. Then the blunt-ended 0.6 kbp SpeI-EcoRV fragment, containing the HCMV promoter, was deleted and replaced with a 0.8 kbp blunt-ended HindIII-EcoRV fragment of pPY22 (kindly provided by Dr. P. Yeh (Villejuif, France), containing the GRE5 promoter, which is inducible by dexamethasone (Mader and White, 1993). A plasmid with correct GRE5-ORF α4 orientation was selected based on restriction enzyme analysis, and was designated pGRINA. This plasmid contains the α4 ORF under the control of GRE5 promoter, followed by the ECMV IRES, the neomycin phosphotransferase ORF and the BGH polyadenylation signal.

Construction of pGemZEO

The prokaryotic EM7-Zeo-pA gene was amplified by PCR from plasmid pZeoSVLacZ (Cayla, Toulouse, France) using the primers (5′ATT CAC TAG TGT ACG GTG GGA GGT CTA TA 3′) and (5′ TCT AGT TTA AAC ACC CTA ACT GAC ACA CAT T 3′), introducing respectively a SpeI and a PmeI site in the amplification product. The PCR product was then cloned into the plasmid pGEM-T (Promega).

Construction of Cosmids Cos6ΔJ and Cos48ΔJ

As a preliminary step, we deleted the two AseI sites that are present in the vector sequences of cosmids cos6 and cos48 (Cunningham and Davison, 1993). To this end, the superCosI vector plasmid was reisolated from cos6 by PacI digestion and the 7.2 kbp PacI-PacI vector fragment was self-ligated. Then, a 0.8 kbp non-essential HpaI-SmaI fragment, containing one AseI site, was excised from superCosI DNA. The second AseI site, located in the ampicilline-resistance gene, was inactivated by AseI digestion and blunt-end ligation of the 1.4 kbp AvaI-EcoRI fragment of pBR327, which contains the tetracycline-resistance gene. The resulting superCosI modified vector, named cosΔ2, was then used to clone the HSV-1 sequences from cos6 and cos48. To this end, the 40.7 kbp and 37.2 kbp herpetic DNA fragments were recovered from cos6 and cos48 by PacI digestion and were inserted into the unique PacI site of cosΔ2, creating cosΔ2-6 and cosΔ2-48 respectively. Then, the 0.8 kbp ApaI-NotI fragment, containing the prokaryotic EM7-Zeo-pA gene was excised from pGemZEO and, after blunt-ending, was inserted into the blunt-ended unique AseI site of cosΔ2-6 and cosΔ2-48, which is located between the HSV-1 genes encoding ICP0 and γ34.5 proteins. Cosmids containing the insert in the required orientation (the PmeI site at the 3′end of EM7-Zeo-pA gene should be adjacent to the γ34.5 gene 3′ end) were identified by digestion and named cos6zeo and cos48zeo respectively. In order to delete the 2.7 kbp restriction fragment XmnI-PmeI, containing most of α4 gene, the cleavage-packaging “a” sequences, and the complex locus containing γ34.5 gene, ORF P and ORF O, from cos6zeo and cos48zeo, both cosmids were first digested with PmeI and then partially digested with XmnI. The largest PmeI-XmnI fragments were self-ligated in both cases, thus generating cosmids cos6ΔJ and cos48ΔJ respectively. The final Cos6ΔJ and Cos48ΔJ constructs contain the tetracycline resistance gene, the Zeo gene, conferring resistance to phleomycin, and a deletion encompassing nucleotides 148493 to 1592 (Cos6Δ J) and nucleotides124776 to 129738 (Cos48ΔJ) of the HSV-1 genome.

Construction of pA-MuCMV-LacZ

Amplicon plasmid pA-SK (Tsitoura et al., 2002), containing one HSV-1 packaging signal (“a”), one HSV-1 origin of replication (ori-S), one multiple cloning site (MCS1) upstream from the “a” signal and a second multiple cloning site (MCS2) downstream of the HSV-1 IE4 promoter, was used to derive the amplicon plasmid pA-MuCMV-LacZ. Firstly, the BamHI-XhoI DNA fragment bearing the EGFP coding region and the bovine growth hormone polyadenylation signal of pIRES-GFP (Clontech) was blunt-ended and cloned at the blunt-ended BamHI site of MCS2, under the control of IE4 promoter. The resulting plasmid was called pA-EUA1. A HindIII transcription unit cassette containing the murine cytomegalovirus enhancer/promoter, the full-length E. coli β-galactosidase ORF and the simian virus 40 polyadenylation signal, was generated by cloning the blunt-ended SmaI-NotI LacZ fragment from pCMV (Clontech) into the SmaI site of pMCMV3 (a gift from Dr. M. Messerle, Max von Pettenkofer-Institute, Muenchen, Germany). Finally, this cassette was cloned into the HindIII site at the MCS1 of pA-EUA1, generating pA-MuCMV-LacZ amplicon plasmid. This plasmid thus contains two independent transcription units expressing EGFP and LacZ reporter proteins.

Construction of ICP4 Expressing Cell Lines

BHK-CINA6 Cells

This cell line was obtained by transfection of sub-confluent BHK-21 cells with 1 μg of pCINA using Effectene (Qiagen, Hilden, Germany). G418 selection (1000 μg/ml) was carried out 48 h after transfection and continued for 3 to 4 weeks, until single isolated colonies were formed. The BHK-CINA6 clone was chosen for its capacity to transcomplement the ICP4 minus D30EBA strain of HSV-1. This cell line was propagated in DMEM supplemented with 10% FBS, 10% TPB and antibiotic.

TE CRE-GRINA129 Cells

To generate cells co-expressing the viral protein ICP4. and Cre recombinase, TE CRE30 cells, expressing Cre recombinase (Logvinoff and Epstein, 2000b), were transfected with 1 μg of pGRINA using Effectene (Qiagen). Two days after transfection, G418 (400 μg/ml) and dexamethasone (10 ng/ml) were added to the medium. After 3 to 4 weeks, individual G418 resistant colonies were isolated and amplified. Colonies were screened for ability to support replication of HSV-1 D30EBA. The cell line TE CRE-GRINA129, which expresses high levels of ICP4, was retained for further study. This cell line was propagated in DMEM supplemented with 10% FBS and antibiotics.

Construction of HSV-1 LaLΔJ Virus

Modified cosmids cos6ΔJ, cos48ΔJ, and cos56LaL, which carries the “a” sequence flanked by two parallel loxP sites in the UL44 locus (Logvinoff and Epstein, 2000b), as well as the non-modified set C cosmids cos28 and cos14, were digested by PacI. DNA was extracted with 1:1 (vol/vol) phenol: chloroform, ethanol precipitated and resuspend in H₂O. A mixture of one microgram of each digested cosmids was used to transfected M64A cells using Effectene (Qiagen) following the manufacturer instructions. The day following transfection, medium was replaced by DMEM supplemented with 10% TBP, 1% FBS and 1% carboxymethylcellulose. Three days later individual plaques were collected and were further purified by three rounds of limit dilution in M64A cells. The structure of several cloned viruses was analyzed by Southern blots, and one of them was chosen for further use in this study, and named HSV-1 LaLΔJ. After the construction of the BHK-CINA6 cell line, HSV-1 LaLΔJ was further purified by three rounds of limit dilution and amplified in these cells

Extraction of Viral DNA from Cytoplasm of Infected Cells

Infected cells were scrapped, pelleted (5 min at 201 g) and washed twice with PBS. The cell pellet was resuspended in hypotonic lysis buffer (10 mM Tris pH8, 10 mM EDTA, 1% NP40, 0.5% deoxycholate) for 10 min in ice. The nuclei were pelleted (20 min at 805 g) and phenol/phenol: chlorophorm extractions were performed on the supernatant, which contains only the packaged viral DNA.

Southern Blots Analysis of Viral DNA

Viral DNA was digested with BamHI and subjected to electrophoresis in 0.7% agarose gel. The DNA samples in gel were UV-depurinated, denaturated, neutralized and transferred to N+ nylon filters (Amersham, Little Chalfort Burckinghamshire, UK), using a vacuum blotting system (Amersham). Probes included a NotI-NotI fragment of pLaL (Logvinoff and Epstein, 2000a), containing a loxP-“a”-loxP sequence, EcoRI-EcoRI α4 fragment from pGemICP4, and SnaBI-AseI α0 fragment from cos6. Probe labeling and hybridizations were performed using the AlkaPhos Direct DNA labeling and CDP star detection system (Amersham) according to the manufacter's protocol.

Preparation of Cell Extracts for Western Blot Analysis

BHK-21 or BHK-CINA6 cells seeded in 24 well plates were either mock infected or exposed to 10 PFU of viruses per cell and maintained at 34° C. in medium 199 supplemented with 1% FBS. At 24 hours post-infection, cells were washed twice in PBS, then harvested and resuspended in 50 μl of H₂O containing a protease inhibitor cocktail (Roche, Indianapolis, Ind. USA). After protein measurment using the Bradford method, 15 μg of proteins were diluted in lysis buffer (62.5 mM Tris HCl pH 6.8; 1% SDS; 0.1 M ditiothreitol; 10% glycerol 0.001% bromophenol blue) and lysed by boiling 5 min.

Western Blot Analysis

Protein samples were separated by electrophoresis in an 8% poly-acrylamide 0.1% SDS gel to detect ICP0 and ICP4 proteins or in an 12% poly-acrylamide 0.1% SDS gel to detect ICP34.5 and US11 proteins. The separated proteins were transferred to a Protran nitrocellulose membrane (Schleicher and Schuell, Dassel, Germany) in Tris-glycin buffer using a Bio-Rad mini-transblot appartus (Bio-rad, München, Germany). Blots were probed with primary antibodies in TBS-T (25 mM Tris, 140 mM NaCl supplemented with 0.1% tween 20) and 5% dry nonfat milk followed by horseradish peroxidase (HRP)-conjugated secondary antibody (DAKO, Glostrup, Denmark) All blots were visualized by ECL plus reagent (Amersham) as directed by the manufactured.

Antibodies

Mouse monoclonal antibodies to ICP4 (clone 8.F.137B US Biological, Swampscott, Mass. USA) were used at 1:4000 dilution. Rabbit polyconal antibodies to ICP0 (Ab 11060, kindly provided by R. D. Everett, MRC Virology Unit, Glasgow, U.K), to ICP34.5 (kindly provided by B. Roizman, University of Chicago, Chicago, U.S.A) and to US11 (kindly provided by J. J. Diaz, University of Lyon, France) (Diaz et al., 1993) were used respectively at 1:10000; 1:3000 and 1:10000 dilutions.

Preparation of Amplicon Vectors

To prepare amplicon stocks we set-up a two-step protocol. In the fist step, BHK-CINA6 cells were plated at a density of 8×10⁵ cells per 60-mm-diameter tissue culture dish and incubated overnight at 37° C. The following day, cells were transfected with 1 μg of pA-MuCMV-LacZ amplicon DNA using LipofectAMINE Plus reagent (Invitrogen) according to manufacter's protocol. One day later, cells were superinfected with HSV-1 LaLΔJ virus at a multiplicity of infection (MOI) of 0.25 in medium 199 supplemented with 1% FBS. Cells were incubated for two additional days at 34° C. before being harvested. Cells were disrupted and virus particles were released using a water-bath sonicator. Cell debris was pelleted (5 min of centrifugation at 2236 g) and the supernatant was recovered and stored at −80° C. Titers of HSV-1 LaLΔJ helper particles were determined by plaque assay in E5 and VERO cells (Berthomme et al., 1995). To titrate amplicon vectors expressing β-galactosidase, Gli36 cells were infected with serial dilutions of viral stock and 24 h later, following fixation and X-gal staining, the number of blue cells were scored. In the second step, the vector stocks, containing amplicon and helper particles, were used to infect TE CRE GRINA129 cells at a MOI of 1 amplicon vector per cell in medium 199 supplemented with 1% FBS. Two days later cells were harvested and sonicated. Amplicon vector and helper virus titers were determined as described above.

X-Gal Staining

Infected cells were fixed for 20 min at 4° C. with formaldehyde 1%, glutaraldehyde 0.2%, and NP40 0.02% in phosphate-buffered saline (PBS). Cells were then washed three times with PBS and stained with PBS solution containing 5 mM ferrocyanine, 5 mM ferricyanide, 2 mM MgCl₂ and 0,05 mg/ml X-Gal (Invitrogen).

Flow Cytometry Studies

Confluent G16.9 cells seeded in 24 well plaque (2×10⁵ cells per well) were infected either with amplicon stock with or without helper virus, or mock infected. All infections were done at a MOI of 5 amplicon vectors or virus per cell, in medium 199 supplemented with 1% FBS at 34° C. Two hours later, cells were washed three times with PBS and incubated in medium 199 supplemented with 1% FBS at 34° C. Two days post-infection, cells were trypsinized, pelleted 5 min at 805 g, and resuspended in PBS supplemented with 1 μg/ml of propidium iodide (PI). In order to determine dead cells (PI-fluorescence) and transduced cells (GFP-fluorescence), cells were analyzed in FACSCalibur flow cytometer (Becton Dickinson, San Jose, Calif.) using Cell Quest software (Becton Dickinson).

.2. Results

Construction of HSV-1-LaLΔJ Helper Virus

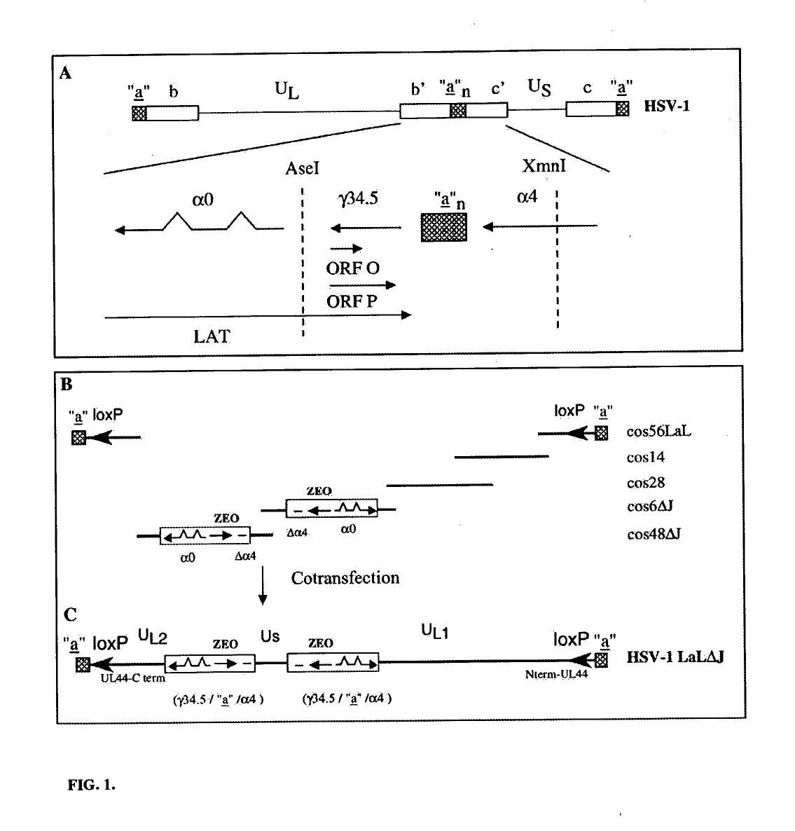

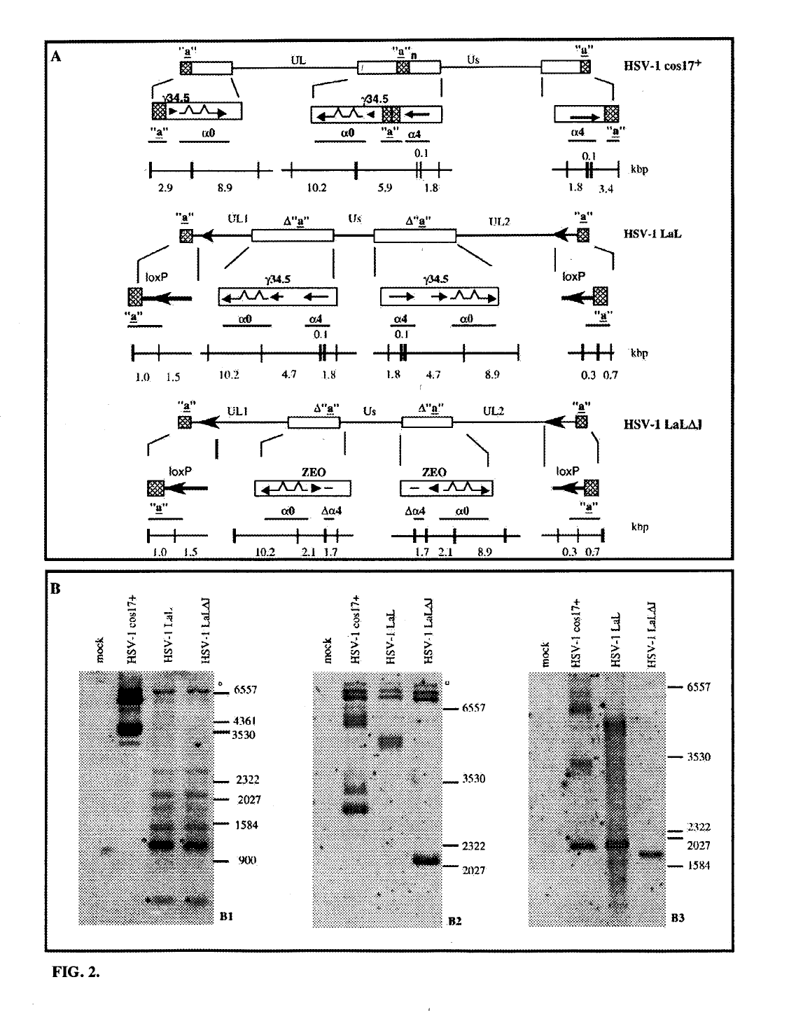

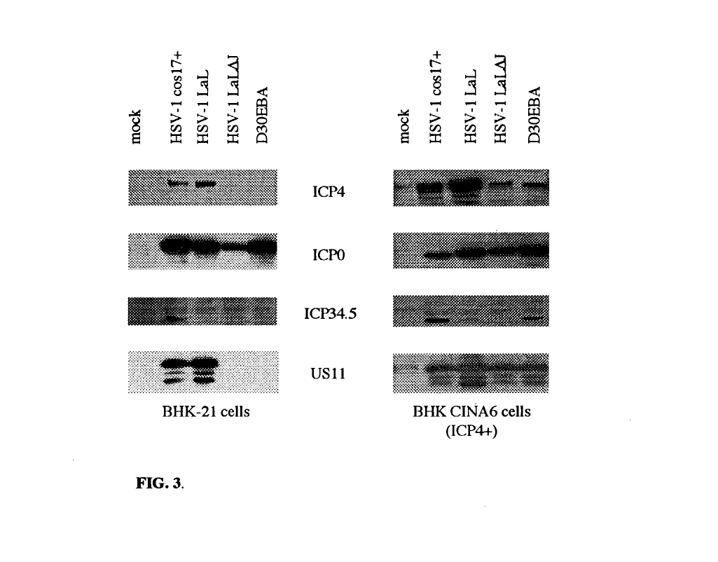

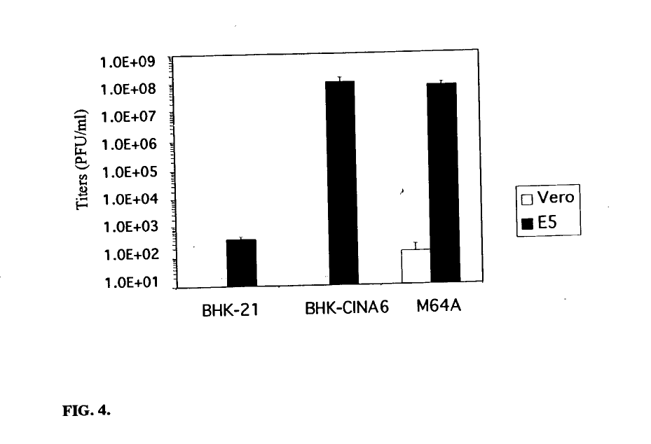

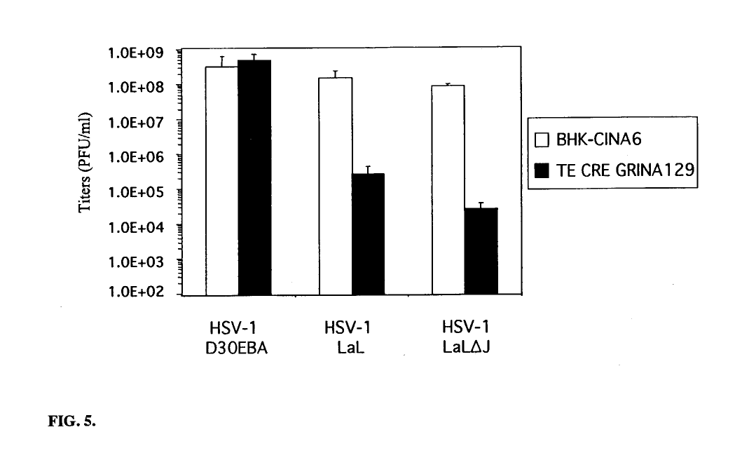

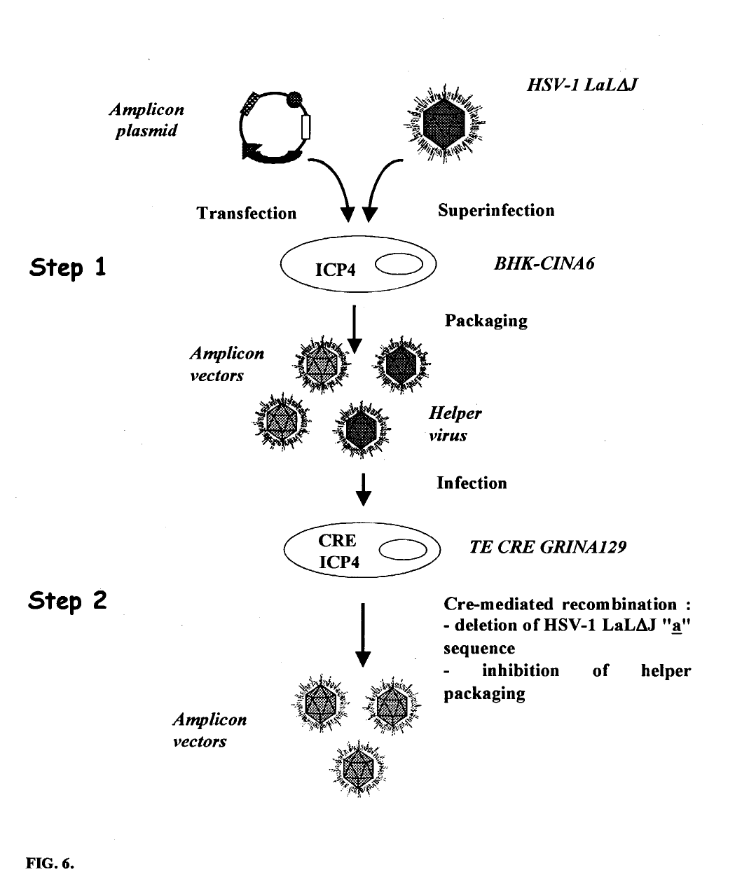

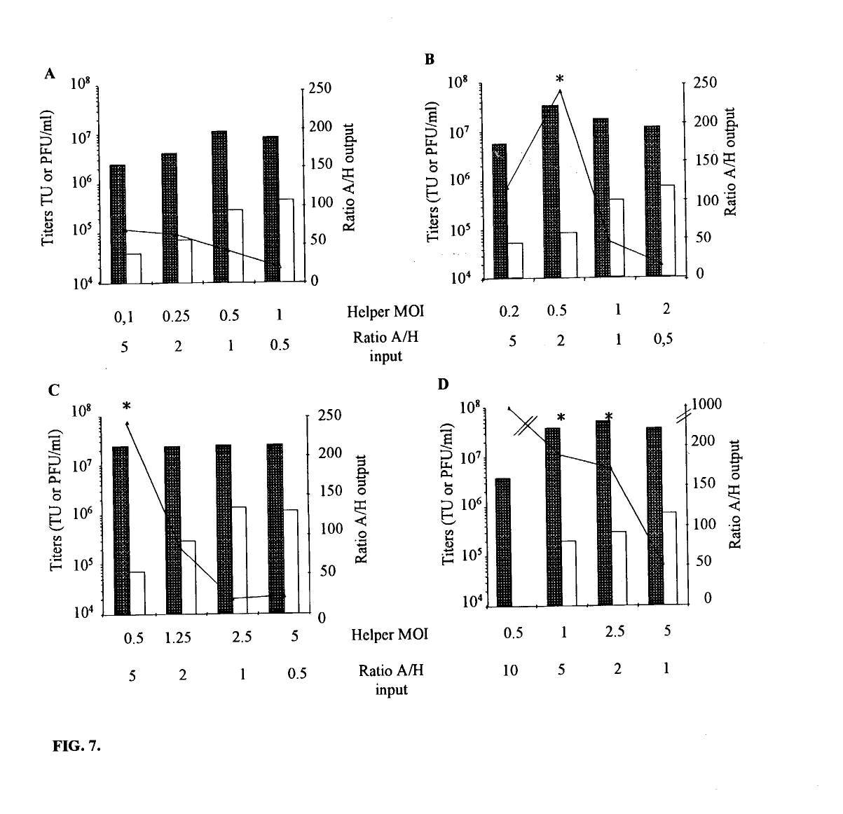

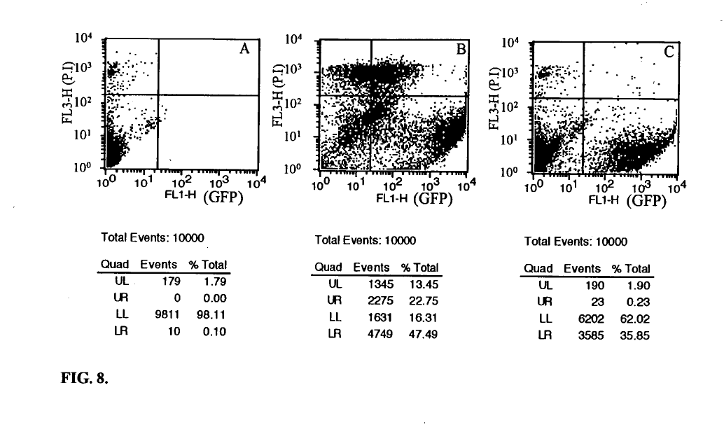

To generate a safe, non-pathogenic helper virus, we decided to delete α4 gene, encoding the essential ICP4 protein. In the absence of ICP4, the virus cycle will be stopped very soon after infection and will not disseminate to other cells. In order to add a second safety barrier, we have also deleted the γ34.5 gene, encoding a protein required for full virulence. To construct this virus, named HSV-1-LaLΔJ, we deleted the major part of the two L-S junctions (nucleotides 124776 to 129738 and 148493 to 1592 in the circular or concatemeric configuration of the HSV-1 genome). Each deleted region (FIG. 1A) spans the γ34.5, ORF P and ORF 0 genes, the 3′ end of the LAT locus, the cleavage-packaging “a” sequences, and the 2700 bps of the 3′ end of the α4 gene. As described in Material and Methods, cosmids cos6ΔJ and cos48ΔJ were cotransfected together with cos56LaL, which contains the floxed “a” sequence into UL44 gene (Logvinoff and Epstein, 2000b), cos14 and cos28 (FIG. 1B) into M64A cells. At 3 days post-transfection, individual plaques were isolated and virus clones were further purified by three rounds of limit dilution in the same cells. After having constructed and characterized the ICP4 expressing BHK CINA6 cell line (see chapter below), a clone of HSV-1 LaLΔJ was further purified by three rounds of limit dilution and virus stocks were amplified in these cells.

The genome of the virus HSV-1 has a reference size Sr of 153 kbp. The genome of the virus HSV-1 LaLΔJ has a size S of 144 kbp. Thus, S=0.94.Sr.

Since the cleavage-packaging “a” sequence is located in the ectopic UL44 locus, the packaged HSV-1 LaLΔJ DNA, like the packaged DNA of HSV-1 LaL (Logvinoff and Epstein, 2000b), is expected to be cleaved at the UL 44 locus, and not at the L-S junctions, thus producing permutations of blocks of genes and cutting the UL region of HSV-1 genome into two separate subregions, UL1 and UL2 (FIG. 1C).

The genomic structure of HSV-1 LaLΔJ was analyzed by hybridization of BamHI-digested HSV-1 LaLΔJ DNA, using a probe containing the “a” signal. HSV-1 cos17+ and HSV-1 LaL BamHI digested DNA were used as controls. As shown in FIG. 2 (2A and 2B1), the pattern of HSV-1 LaLΔJ virus differs from that of HSV-1 cos 17+, but is similar to that of HSV-1 LaL, with no internal “a” sequences. It presents two novel genomic ends of 0.7 kpb and 1 kpb (labeled with asterisks) and includes a family of bands with 500 bp increments corresponding to amplifications of the “a” sequence (Logvinoff and Epstein, 2000a).

To confirm the deletions at the L-S junctions, BamRHI-digested DNAs were further hybridized with different probes. Hybridization with an α0 probe revealed two fragments of 10.2 kbp and 8.9 kbp corresponding respectively to UL-b′ and UL-b junctions, which are common to the three viruses. In contrast, the 2.9 kbp and 5.9 kbp bands corresponding to ab and b′a′c′ sequences, containing also the γ34.5 gene, were observed only for HSV-1 cos17+. As HSV-1 LaL lacks the two copies of the native “a” sequence, the 2.9 kbp and 5.9 kbp fragments were replaced by two copies of a 4.7 kbp fragment (see FIGS. 2A and 2B-2). For HSV-1 LaLΔJ, the 4.7 kbp fragments shifted to 2.1 kbp due to deletion of both copies of γ34.5 gene and their replacement by the Zeo gene, which contains one additional BamHI site (FIGS. 2A and 2B-3). The faint fragment of 3.4 kbp that can be observed in HSV-1 cos17+ corresponds to an increment of 500 bp to the 2.9 kpb fragment, which is characteristic of “a” sequence duplication at the UL end. Duplication of “a” sequence at the L-S junction could not be detected at the separation range of agarose gel used.

Lastly, the deletion of 2.7 kbp at the 3′ end of α4 gene was confirmed by hybridization with an α4 probe (FIGS. 2A and 2B-3). As expected, α4 probe detected three fragments of 5.9 kbp, 3.4 kbp and 1.8 kbp in HSV-1 cos17+genome, two fragments of 4.7 kbp and 1.8 kbp in HSV-1 LaL genome, while the probe hybridized with only one fragment of 1.7 kbp in HSV-1 LaLΔJ genome. The detection of a single fragment for HSV-1 LaLΔJ DNA confirmed the deletion of the 3′ end of the α4 gene including one BamH1 site. The 1.7 kbp fragment contains the 1.3 kbp of the 5′ end of α4 gene still present in the HSV-1 LaLΔJ genome.

These results confirm [by Southern blots (FIG. 2)] that HSV-1 LaLΔJ has the expected genomic structure, indicating that this virus carries the deletion spanning the γ34.5 locus, the native “a” sequences and the last two thirds of the α4 gene, and that is cleaved at the unique “a” sequence inserted in the UL44 locus.

In order to study the phenotype of HSV-1 LaLΔJ, BHK-21 and BHK-CINA 6 cells were infected at 10 PFU/cell and protein synthesis was analyzed by Western blots using antibodies specific for the immediate early proteins ICP4 and ICP0, and for the late proteins ICP34.5, and US11. As controls, cells were also infected with HSV-1 cos17+, HSV-1 LaL, and HSV-1 D30EBA, a previously described ICP4 minus virus (Paterson and Everett, 1990). As shown in FIG. 3, only the immediate early ICP0 protein was detected in the BHK-21 cells infected with HSV-1 LaLΔJ or HSV-1 D30EBA strains, thus confirming that HSV-1 LaLΔJ, like D30EBA, does not express wild type ICP4 protein and cannot therefore induce synthesis of late (ICP34.5 and US11) proteins. Interestingly we observed no ICP34.5 synthesis in BHK-21 cells infected with HSV-1 LaL, although this virus can express the US11 late protein in these cells. This can most likely be explained by the absence of the ICP34.5 promoter, located in the native “a” sequences (Martin and Weber, 1998) which are absent in HSV-1 LaL (Logvinoff and Epstein, 2000a). In the ICP4 plus BHK CINA6 cells, HSV-1 LaLΔJ induced wild type levels of the late US11 protein, but not of ICP34.5 protein, in contrast to HSV-1 D30EBA, which expressed both proteins. Taken together, these experiments [Western blots of proteins induced by this virus in cells expressing or not ICP4, using specific antibodies] thus confirmed the ICP4 minus/ICP34.5 minus phenotype of HSV-1 LaLΔJ.

It is interesting to note that these studies showed that HSV-1 LaL is also deficient for α34.5 expression.

Construction and Properties of Novel ICP4-Expressing Cell Lines

BHK CINA6 Cells

Most cell lines expressing ICP4, like E5 and M64A cells, contain the entire HSV-1 ICP4 transcription unit and surrounding regions and can thus generate revertant viruses through homologous recombination at the ICP4 locus of defective HSV-1 genomes. In order to produce the novel HSV-1 LaLΔJ helper virus without generating such revertants, we engineered a new cell line, in which the α4 ORF is surrounded by sequences non homologous to HSV-1, as described in Materials and Methods. This cell line, which derives from BHK-21 cells, was named BHK CINA6. To confirm that BHK CINA6 cells are able to transcomplement ICP4 minus virus, we compared the growth HSV-1 LaLΔJ in BHK-21, BHK CINA6, and M64A cells. The virus productions were titrated in E5 cells, which allow plaque formation of ICP4 minus viruses, and in Vero cells, which allow the detection of revertant ICP4 plus virus. As shown in FIG. 4, HSV-1 LaLΔJ grows to high titers both in BHK-CINA6 and in M64A cells, but not in BHK-21 cells, demonstrating that BHK CINA6 cells are able to complement ICP4 minus virus as well as M64A cells. The few plaques observed after infection of BHK-21 cells, when titration was done in E5 cells, correspond, most likely, to residual particles, as in Vero cells no virus was able to form plaques. In addition, BHK CINA6 cells generated no HSV-1 LaLΔJ particles able to form plaques in Vero cells, at the opposite of M64A cells, which generated a small amount of such revertant particles. Growth of HSV-1 LaLΔJ in cells expressing ICP4, were roughly similar to that of HSV-1 D30EBA (data not shown), indicating that the small size (144 kbp) and the atypical structure of HSV-1 LaLΔJ genome do not impair its ability to grow in ICP4 transcomplementing cells. Taken together, these results thus (i) highlight the relevance of developing novel non-recombinogenic complementing cell lines, (ii) confirm the ICP4 minus phenotype of HSV-1 LaLΔJ and (iii) confirm that HSV-1 LaLΔJ stocks produced in BHK-CINA6 cells has no detectable replication competent contaminants.

TE CRE GRINA 129

In order to inhibit cleavage-encapsidation of helper HSV-1-LaLΔJ replication concatemers without impairing their expression, we engineered a cell line expressing both Cre recombinase and ICP4, named TE CRE GRINA129, as described in Materials and Methods. This cell line derives from TE-CRE30 cells, which express only Cre recombinase, and had been previously shown to efficiently inhibit cleavage-encapsidation of HSV-1 LaL virus by leading loxP site-specific recombination and excision of the unique floxed “a” sequence carried by this viral genome (Logvinoff and Epstein, 2000a). To confirm that TE CRE GRINA129 cells simultaneously express ICP4 and Cre recombinase, we compared the growth of HSV-1 D30EBA, HSV-1 LaL, and HSV-1-LaLΔJ in BHK CINA6 and TE CRE GRINA129 cells. As shown in FIG. 5, the Cre-insensitive HSV-1 D30EBA grows equally well in TE CRE GRINA129 cells and in BHK CINA6 cells, confirming the efficiency of ICP4 transcomplementation of TE CREGRINA129 cells. On the other hand, growth of HSV-1 LaL is inhibited by almost 3 logs in these cells, as compared to in BHK CINA6 cells, confirming the efficiency of Cre expression and of site-specific deletion of the floxed “a” sequence, in TE CRE GRINA129 cells. HSV-1-LaLΔJ is also strongly inhibited in these cells, by more than 3 logs, confirming the Cre sensitive phenotype of this virus. As with BHK CINA6 cells, we observed no generation of replication competent particles on TE CREGRINA129 cells (data not shown).

In summary, the results presented in the two last paragraphs confirm (i) that both BHK-CINA6 and TE-CRE GRINA129 cells allow efficient multiplication of ICP4 minus viruses, (ii) that TE CRE GRINA129 cells, in addition, inhibit the production of viruses carrying a “floxed” “a” sequence and (iii) that HSV-1 LaLΔJ has an ICP4 minus and Cre-sensitive phenotype.

Production of Amplicon Vectors Using HSV-1 LaLΔJ Virus, BHK CINA6 Cells and TE CRE GRINA129 Cells

The results described above thus suggested that the novel packaging system composed of HSV-1 LaLΔJ, BHK CINA6 cells and TE CRE GRINA129 cells presented the required characteristics for preparing high amounts of non pathogenic amplicons stocks. This was confirmed by producing amplicon vectors according to the strategy previously described with HSV-1 LaL and TE-CRE30 cells (Logvinoff and Epstein, 2001). Briefly, helper-contaminated amplicon stocks are first produced in BHK-CINA6 by transfection of amplicon plasmid and superinfection with HSV-1 LaLΔJ. In a second step, this stock is further passaged on TE CRE GRINA129 cells to produce vector particles while inhibiting packaging of the helper genomes (FIG. 6). As HSV-1 LaLΔJ cleavage-packaging is strongly inhibited in these cells, the stock of amplicon vectors thus produced is expected to be only slightly contaminated with defective HSV-1 LaLΔJ helper particles.

More precisely, BHK-CINA6 cells were transfected with the amplicon plasmid pA-MuCMV-LacZ, which expresses the E. coli lacZ and GFP reporter proteins under the control of different promoters. The following day, transfected cells were superinfected with HSV-1 LaLΔJ at a MOI of 0.25 PFU/cell and viral production was harvested 48 h after infection and titrated. In average, we obtained 9.5×10 ⁷ transducing units (TU)/ml of amplicon vectors and 2.53×10⁷ PFU/ml of HSV-1 LaLΔJ as shown in Table 1. The ratio of amplicon vectors to HSV-1 LaLΔJ particles after the first step was always in favor of amplicons, and ranged from 2 to 10 in different experiments. In the second step, the helper particles were cleared out by infecting TE CRE GRINA129 cells at a MOI of 1 amplicon vector per cell. Two days later, particles were harvested and titrated. The mean titers obtained after step 2 were 2.6×10⁷ TU/ml for amplicon vectors and 1.16×10⁵ PFU/ml for helper virus, corresponding to a mean amplicon/helper ratio of 224. No revertant helper particles, able to grow in VERO cells, were detected.

To set up the conditions yielding both a high-titer amplicon vector production and a high-ratio of vector to helper particles, TE CRE GRINA129 cells were infected at increasing MOIs of amplicon vectors, using stocks carrying variable vector to helper ratios (in some cases, helper particles were added to the vector stocks to reach the desired ratio). As shown in FIG. 7, the best arrangement (labeled with asterisks) were obtained when infecting at (i) a vector to helper ratio of at least 2 and (ii) a helper virus MOI of 0.5 to 2 PFU/cell. These tests were made at least three times, with rather similar results. Employing this improved protocol, we were able to obtain, after the second step, more than 1×10⁸ total transducing units (with a contamination level of helper particles lower than 0.5%), using at the starting point only one 100-mm-diameter dish of BHK-CINA6 cells. Thus, high amount of amplicon vectors with titers higher than 5×10⁸ TU/ml after concentration could be readily achieved.

Amplicon Stocks Generated Using the Novel System are not Cytotoxic

In order to assess the cytotoxicity of amplicon vectors produced with the novel packaging system, several cell lines (TE671, VERO, Hep-2, HeLa, BHK21, G16.9 and NIH 3T3 cells), were infected at a MOI of 5 amplicons per cell using pA-MuCMV-LacZ amplicon vector stocks containing an amplicon/helper ratio of 3 (obtained after the first step, in BHK-CINA6 cells), or a ratio of 250 (obtained after the second step, in TE CRE GRINA129 cells). Two days post-infection cells were stained with propidium iodide (PI), which is inserted into the DNA of cells that have lost membrane integrity therefore marking cells that are committed to die. Since pA-MuCMV-LacZ amplicon vectors express GFP, we have analyzed the viability of cells infected by amplicon vectors using FACS. Both PI and GFP fluorescence of infected cells are represented in FIG. 8, which shows infection of G16.9 cells. Infection of these cells with the amplicon stock showing the higher level of contamination with helper virus resulted in about 70% of GFP-labeled cells, whereas 36.2% of cells were labeled with PI (FIG. 8B). On the other hand, using the amplicon stock prepared after the second step of the protocol (FIG. 8C) around 36% of cells were GFP-positive. In this case, however, we observed no more PI-labeled cells than in the control non-infected cells (FIG. 8A), indicating that the vector stocks produced in TE CRE GRINA129 cells were not cytotoxic. Very similar results were observed with the other cell lines (data not shown). These results underline the importance of reducing the amount of contaminant helper particles present in the stocks.

TABLE 1

| | | | | Ratio |
|---|---|---|---|---|
| | | Amplicon | Helper virus | amplicon/helper |
| | | (Tu/ml)^{c} | (PFU/ml)^{d} | (TU/PFU) |
| BHK-CINA6^{a} | 1^{e} | 1.1 × 10⁸ | 1.1 × 10⁷ | 10 |
| | 2^{e} | 8.0 × 10⁷ | 2.3 × 10⁷ | 3.5 |
| | 3^{e} | 9.5 × 10⁷ | 4.2 × 10⁷ | 2.3 |
| | Avg | 9.5 × 10⁷ | 2.53 × 10⁷ | 3.75 |
| TE CRE | 1^{e} | 5.0 × 10⁷ | 2.1 × 10⁵ | 238 |
| GRINA129^{b} | 2^{e} | 1.5 × 10⁷ | 8.0 × 10⁴ | 187 |
| | 3^{e} | 1.5 × 10⁷ | 6.0 × 10⁴ | 250 |
| | Avg | 2.6 × 10⁷ | 1.16 × 10⁵ | 224 |

### REFERENCES

BATAILLE, D., and EPSTEIN, A. L. (1997). Equimolar generation of the four possible arrangements of adjacent L components in herpes simplex virus type 1 replicative intermediates. J Virol 71, 7736-7743.

BERTHOMME, H., MONAHAN, S. J., PARRIS, D. S., JACQUEMONT, B., and EPSTEIN, A. L. (1995). Cloning, sequencing, and functional characterization of the two subunits of the pseudorabies virus DNA polymerase holoenzyme: evidence for specificity of interaction. J Virol 69, 2811-2818.

CAREW, J. F., KOOBY, D. A., HALTERMAN, M. W., KIM, S. H., FEDEROFF, H. J., and FONG, Y. (2001). A novel approach to cancer therapy using an oncolytic herpes virus to package amplicons containing cytokine genes. Mol Ther 4, 250-256.

CUNNINGHAM, C., and DAVISON, A. J. (1993). A cosmid-based system for constructing mutants of herpes simplex virus type 1. Virology 197, 116-124.

DAVIDSON, I., and STOW, N. D. (1985). Expression of an immediate early polypeptide and activation of a viral origin of DNA replication in cells containing a fragment of herpes simplex virus DNA. Virology 141, 77-88.

DELMAN, K. A., ZAGER, J. S., BENNETT, J. J., MALHOTRA, S., EBRIGHT, M. I., MCAULIFFE, P. F., HALTERMAN, M. W., FEDEROFF, H. J., and FONG, Y. (2002). Efficacy of multiagent herpes simplex virus amplicon-mediated immunotherapy as adjuvant treatment for experimental hepatic cancer. Ann Surg 236, 337-342; discussion 342-333.

DELUCA, N. A., MCCARTHY, A. M., and SCHAFFER, P. A. (1985). Isolation and characterization of deletion mutants of herpes simplex virus type 1 in the gene encoding immediate-early regulatory protein ICP4. J Virol 56, 558-570.

DELUCA, N. A., and SCHAFFER, P. A. (1988). Physical and functional domains of the herpes simplex virus transcriptional regulatory protein ICP4. J Virol 62, 732-743.

DIAZ, J. J., SIMONIN, D., MASSE, T., DEVILLER, P., KINDBEITER, K., DENOROY, L., and MADJAR, J. J. (1993). The herpes simplex virus type 1 US11 gene product is a phosphorylated protein found to be non-specifically associated with both ribosomal subunits. J Gen Virol 74, 397-406.

FEDEROFF, H. J., GESCHWIND, M. D., GELLER, A. I., and KESSLER, J. A. (1992). Expression of nerve growth factor in vivo from a defective herpes simplex virus 1 vector prevents effects of axotomy on sympathetic ganglia. Proc Natl Acad Sci U S A 89, 1636-1640.

FRAEFEL, C., SONG, S., LIM, F., LANG, P., YU, L., WANG, Y., WILD, P., and GELLER, A. I. (1996). Helper virus-free transfer of herpes simplex virus type 1 plasmid vectors into neural cells. J Virol 70, 7190-7197.

GELLER, A. I., and BREAKEFIELD, X. O. (1988). A defective HSV-1 vector expresses Escherichia coli beta-galactosidase in cultured peripheral neurons. Science 241, 1667-1669.

GELLER, A. I., KEYOMARSI, K., BRYAN, J., and PARDEE, A. B. (1990). An efficient deletion mutant packaging system for defective herpes simplex virus vectors: potential applications to human gene therapy and neuronal physiology. Proceedings of the National Academy of Sciences of the United States of America 87, 8950-8954.

HO, D. Y., MOCARSKI, E. S., and SAPOLSKY, R. M. (1993). Altering central nervous system physiology with a defective herpes simplex virus vector expressing the glucose transporter gene. Proc Natl Acad Sci USA 90, 3655-3659.

HOCKNELL, P. K., WILEY, R. D., WANG, X., EVANS, T. G., BOWERS, W. J., HANKE, T., FEDEROFF, H. J., and DEWHURST, S. (2002). Expression of human immunodeficiency virus type 1 gp120 from herpes simplex virus type 1-derived amplicons results in potent, specific, and durable cellular and humoral immune responses. J Virol 76, 5565-5580.

JOHNSON, P. A., MIYANOHARA, A., LEVINE, F., CAHILL, T., and FRIEDMANN, T. (1992). Cytotoxicity of a replication-defective mutant of herpes simplex virus type 1. J Virol 66, 2952-2965.

KASHIMA, T., VINTERS, H. V., CAMPAGNONI, A. T., (1995). Unexpected expression of intermediate filament protein genes in human oligodendroglioma cell lines. J. Neuropathol. Exp. Neurol 54, 23-31.

KWONG, A. D., and FRENKEL, N. (1985). The herpes simplex virus amplicon. IV. Efficient expression of a chimeric chicken ovalbumin gene amplified within defective virus genomes. Virology 142, 421-425.

LEIB AND OLIVO, BioEssays 15:547-554, 1993

LIM, F., HARTLEY, D., STARR, P., LANG, P., SONG, S., YU, L., WANG, Y., and GELLER, A. I. (1996). Generation of high-titer defective HSV-1 vectors using an IE 2 deletion mutant and quantitative study of expression in cultured cortical cells. Biotechniques 20, 460-469.

LOGVINOFF, C., and EPSTEIN, A. L. (2000a). Genetic engineering of herpes simplex virus and vector genomes carrying loxP sites in cells expressing Cre recombinase. Virology 267, 102-110.

LOGVINOFF, C., and EPSTEIN, A. L. (2000b). Intracellular Cre-mediated deletion of the unique packaging signal carried by a herpes simplex virus type 1 recombinant and its relationship to the cleavage-packaging process. J Virol 74, 8402-8412.

LOGVINOFF, C., and EPSTEIN, A. L. (2001). A novel approach for herpes simplex virus type 1 amplicon vector production, using the Cre-loxP recombination system to remove helper virus. Hum Gene Ther 12, 161-167.

MADER, S., and WHITE, J. H. (1993). A steroid-inducible promoter for the controlled overexpression of cloned genes in eukaryotic cells. Proc Natl Acad Sci USA 90, 5603-5607.

MARTIN, D. W., and WEBER, P. C. (1998). Role of the DR2 repeat array in the regulation of the ICP34.5 gene promoter of herpes simplex virus type 1 during productive infection. J Gen Virol 79, 517-523.

PATERSON, T., and EVERETT, R. D. (1990). A prominent serine-rich region in Vmw175, the major transcriptional regulator protein of herpes simplex virus type 1, is not essential for virus growth in tissue culture. J Gen Virol 71, 1775-1783.

SAEKI, Y., FRAEFEL, C., ICHIKAWA, T., BREAKEFIELD, X. O., and CHIOCCA, E. A. (2001). Improved Helper Virus-Free Packaging System for HSV Amplicon Vectors Using an ICP27-Deleted, Oversized HSV-1 DNA in a Bacterial Artificial Chromosome. Mol Ther 3, 591-601.

SAEKI, Y., ICHIKAWA, T., SAEKI, A., CHIOCCA, E. A., TOBLER, K., ACKERMANN, M., BREAKEFIELD, X. O., and FRAEFEL, C. (1998). Herpes simplex virus type 1 DNA amplified as bacterial artificial chromosome in Escherichia coli: rescue of replication-competent virus progeny and packaging of amplicon vectors. Hum Gene Ther 9, 2787-2794.

SPAETE, R. R., and FRENKEL, N. (1982). The herpes simplex virus amplicon: a new eucaryotic defective-virus cloning-amplifying vector. Cell 30, 295-304.

STAVROPOULOS, T. A., and STRATHDEE, C. A. (1998). An enhanced packaging system for helper-dependent herpes simplex virus vectors. J Virol 72, 7137-7143.

SUN, M., ZHANG, G. R., YANG, T., YU, L., and GELLER, A. I. (1999). Improved titers for helper virus-free herpes simplex virus type 1 plasmid vectors by optimization of the packaging protocol and addition of noninfectious herpes simplex virus-related particles (previral DNA replication enveloped particles) to the packaging procedure. Hum Gene Ther 10, 2005-2011.

TSITOURA, E., LUCAS, M., REVOL-GUYOT, V., EPSTEIN, A. L., MANSERVIGI, R., and MAVROMARA, P. (2002). Expression of hepatitis C virus envelope glycoproteins by herpes simplex virus type 1-based amplicon vectors. J Gen Virol 83, 561-566.

WADE-MARTINS, R., SMITH, E. R., TYMINSKI, E., CHIOCCA, E. A., and SAEKI, Y. (2001). An infectious transfer and expression system for genomic DNA loci in human and mouse cells. Nat Biotechnol 19, 1067-1070.

WILLIS, R. A., BOWERS, W. J., TURNER, M. J., FISHER, T. L., ABDUL-ALIM, C. S., HOWARD, D. F., FEDEROFF, H. J., LORD, E. M., and FRELINGER, J. G. (2001). Dendritic cells transduced with HSV-1 amplicons expressing prostate-specific antigen generate antitumor immunity in mice. Hum Gene Ther 12, 1867-1879.

1- Method for producing non-pathogenic defective amplicon vectors derived from herpes viridae species by means of an helper system comprising at least one kind of cells and at least one kind of helper virus which is finally at least partially deleted by means of a site-specific recombination system involving the packaging signals “a” of the helper virus in the cells where the amplicon vectors are produced,
said method including notably the following essential steps:
-a- transfection of cells C1 from a first cell line by the amplicon vectors;
-b- (super)infection of said cells C1 with the helper virus;
-c- culture of transfected and (super)infected cells C1;
-d- harvest of the so produced amplicon vectors and helper virus;
-e- infection of cells C2 from a second cell line different from C1, by at least one part of the harvested amplicon vectors and helper virus;
-f- culture of infected cells C2;
-g- harvest of the so produced particles of the amplicon vectors free or substantially free of helper virus;
wherein:
(i) the helper virus's recombinant genome has preferably a size S (kbp) defined as follows with respect to the reference size Sr (kbp) of the virus's helper genome free from any deletion of coding sequence(s) encoding for at least one protein essential for viral production of the helper virus:

| | |
|---|---|
| | S ≦ 0.99 · Sr |
| preferably | S ≦ 0.95 · Sr |
| more preferably | S ≦ 0.90 · Sr |

(ii) the helper virus's recombinant genome includes a packaging specific site recognizable and deletable by cells C2;
(iii) the infection -e- of cells C2 by the helper virus results in deletion of the packaging signal(s) “a”, said deletion so involving an additional size reduction;
(iv) the helper virus's recombinant genome is totally or partially defective in coding sequence(s) encoding for at least one essential protein (Pe) and eventually at least one non-essential protein (Pne) for viral production of the helper virus;
(v) the cells C1 and C2 are able to transcomplement at least one of the essential protein(s) Pe and eventually at least one of the non-essential protein(s) (Pne) and are so able to make up for the genomic deficiency of the helper virus;
(vi) and the cells C2 are able to recognize and to delete the packaging specific site “a” of the helper virus.

2- Method according to claim 1 wherein the helper virus's recombinant genome is subjected
to a first size reduction corresponding to the deletion of the coding sequence(s) encoding for at least one protein essential (Pe) and eventually at least one non-essential protein (Pne) for viral production of the helper virus, said first size reduction occurring before cells C1 & C2 (super)infections,
and to second size reduction corresponding to the deletion of the packaging specific site “a” of the helper virus, in the cells C2;
so that the helper virus encapsidation be prevented.

3- Method according to claim 1 wherein the site-specific recombination system involving the packaging signals “a” of the helper virus, comprises at least one enzyme specific of at least one sequence delimited by 2 identical sites, said system being preferably selected in the group including enzyme Cre/sites loxP-“a”-loxP and enzyme Flp/sitesfrp-“a”-frp.

4- Method according to claim 1 wherein the helper virus's recombinant genome contains at least one (preferably a single) floxed “a” packaging signal located in non-essential loci, preferably in gC locus.

5- Method according to claim 1 wherein at least part of the coding sequence(s) encoding for one essential protein (Pe₁) and one non-essential protein (Pne₁) are lacking in the helper virus's recombinant genome, Pe₁ and Pne₁ being preferably selected in the ICP proteins group, and more preferably Pe₁ being ICP4 and Pne₁ being ICP34.5.

6- Method according to claim 1 wherein the final residual virus helper particles concentration is inferior or equal to 0.5%, preferably to 0.3%, and more preferably to 0.2% of the produced viral population.

7- Method according to claim 1 wherein Sr is comprised between 10 to 500 kbp, preferably between 50 to 300 kbp, and more preferably between 100 to 200 kbp.

8- Method according to claim 1 wherein the amplicon plasmid contains at least one gene of neurobiological, immunologic or therapeutic interest.

9- Method according to claim 1 wherein the amplicon plasmid is pA-MuCMV-LacZ, as defined in the instant specification and enclosed figures.

10- Method according to claim 1 wherein the helper virus is HSV-1 LaLΔJ, C1 are BHK-CINA6 cells and C2 are TE CRE GRINA129 cells, as defined in the instant specification and enclosed figures.

11- Defective helper virus belonging to herpes viridae species, notably useful for producing non-pathogenic defective amplicon vectors derived from herpes viridae species, said virus comprising a recombinant genome:
(i) which size S (kbp) is preferably defined as follows with respect to the reference size Sr (kbp) of the virus's helper genome free from any deletion of coding sequence(s) encoding for at least one protein essential for viral production of the helper virus:

| | |
|---|---|
| | S ≦ 0.99 · Sr |
| preferably | S ≦ 0.95 · Sr |
| more preferably | S ≦ 0.90 · Sr |

(ii) including a packaging specific site recognizable and deletable by appropriated cells named C2;
(iii) and being totally or partially defective in coding sequencers) encoding for at least one protein essential (Pe) for the production of the helper virus.

12- Defective helper virus according to claim 11 which genome comprises at least one sequence including the packaging signals “a” flanked by 2 identical sites, these latter being selected in the group including sites loxP and sites frp, said sequence being specifically attacked by an enzyme selected in the group including Cre and Flp.

13- Defective helper virus according to claim 11 which recombinant genome contains at least one (preferably a single) floxed “a” packaging signal located in a non-essential locus, preferably in gC locus.

14- Defective helper virus according to claim 11 which wherein at least part of the coding sequence(s) encoding for one essential protein (Pe₁) and one non-essential protein (Pne₁) are lacking in the helper virus's recombinant genome, Pe₁ and Pne₁ being preferably selected in the ICP proteins group, and more preferably Pe₁ being ICP4 and Pne₁ being ICP34.5.

15- Defective helper virus according to claim 11, wherein Sr is comprised between 10 to 500 kbp, preferably between 50 to 300 kbp, and more preferably between 100 to 200 kbp.

16- Defective helper virus according to claim 11 consisting of HSV-1 LaLΔJ, as defined in the instant specification and enclosed figures.

17- Recombinant genome of the defective helper virus according to claim 11, its transcription products and its traduction products.

18- Cells C1 or C2 which are able to transcomplement at least one of the essential protein(s) Pe of the defective helper virus according to claim 11 and are so able to make up for the genomic deficiency of said defective helper virus.

19- Cells C1 or C2 according to claim 18, wherein there are one essential protein Pe₁ and one non-essential protein Pne₁, Pe₁ and Pne₁ being preferably selected in the ICP proteins group, and more preferably Pe₁ being ICP4 and Pne₁ being ICP34.5.

20- Cells C2 which are able to recognize and to delete the packaging specific site “a” of the helper virus according to claim 11.

21- Cells C1 which consist of BHK-CINA6 cells, as defined in the instant specification and enclosed figures.

22- Cells C2 which consist of TE CRE GRINA129 cells, as defined in the instant specification and enclosed figures.

23- Transfected cells C1 and/or (super)infected cells C1 obtained by the method according to claim 1.

24- Infected cells C2 obtained by the method according to claim 1.

25- Helper system for producing non-pathogenic defective amplicon vectors derived from herpes viridae species, said system comprising at least one defective helper virus according to claim 11, cells C1 and cells C2.

26- Method for the production of a defective helper virus belonging to herpes viridae species, notably useful for producing non-pathogenic defective amplicon vectors derived from herpes viridae species, consisting essentially in
I—constructing a recombinant genome:
free from any native packaging specific site “a”
including a packaging specific site recognizable and deletable by appropriated cells named C2;
II—and reducing the size of the genome so as to obtain a size S which contributes at least partially to prevent the helper virus encapsidation.

27- Method according to claim 26 wherein
the construction step -I- consists essentially in:
deleting the native packaging specific site “a” of the helper virus,
inserting into the helper virus genome a single “a” packaging signal located in non-essential loci, preferably in gC locus, said packaging signal “a” being flanked by 2 identical sites, these latter being selected in the group including sites loxP and sites frp, said sequence being specifically attackable by an enzyme selected in the group including Cre and Flp,
and the size reduction step -II- consists essentially in deleting in the recombinant genome, at least part of the coding sequence(s) encoding one essential protein Pe₁ and one non-essential protein Pne₁, Pe₁ and Pne₁ being preferably selected in the ICP proteins group, and more preferably Pe₁ being ICP4 and Pne₁ being ICP34.5, so that the size S (kbp) of the recombinant genome be defined as follows with respect to the reference size Sr (kbp) of the virus's helper genome free from any deletion of coding sequence(s) encoding for at least one protein essential for viral production of the helper virus:

| | |
|---|---|
| | S ≦ 0.99 · Sr |
| preferably | S ≦ 0.95 · Sr |
| more preferably | S ≦ 0.90 · Sr. |

28- Method of treating a patient comprising administering to the patient an HSV amplicon vectors obtained by the method according to claim 1.

29- Method of treating a patient comprising administering to the patient the infected cells of claim 24.

30- Drugs for gene therapy comprising the HSV amplicon vectors obtained by the method according to claim 1.
